(19) United States
(12) Patent Application Publication
(10) Pub. No.: US 2026/0098790 A1
(43) Pub. Date: Apr. 9, 2026

(54) REAGENT-COATED COVER SLIP

(71) Applicant: Bonraybio Co., Ltd., Taichung City (TW)

(72) Inventors: Cheng-Teng Hsu, Taichung City (TW); Hui-Chen Chang, Taichung City (TW); Ying-Jyh Liao, Taichung City (TW)

(21) Appl. No.: 19/406,126

(22) Filed: Dec. 2, 2025

Related U.S. Application Data
(63) Continuation-in-part of application No. PCT/US2024/034356, filed on Jun. 17, 2024.
(60) Provisional application No. 63/521,866, filed on Jun. 19, 2023.
(60) Provisional application No. 63/562,960, filed on Mar. 8, 2024.
(60) Provisional application No. 63/729,909, filed on Dec. 9, 2024.
(60) Provisional application No. 63/738,401, filed on Dec. 23, 2024.

(51) Int. Cl. G01N 1/31; G01N 1/30; G01N 33/483

(52) U.S. Cl. CPC G01N 1/312; G01N 1/30; G01N 33/4833

(57) ABSTRACT

The present invention relates to reagent-coated cover slips and methods of using them to carry out one or more reactions. The present invention provides methods using reagent-coated cover slips to carry out a test on a carrier slide. The present invention provides methods where sample cells are immobilized on a carrier slide and a test is carried on the carrier slide. The present methods carry out one or more chemical reactions in the embedded sample cells on a carrier slide by contacting reagent-coated cover slips with sample cells immobilized on the carrier slide to result in a reaction product on the carrier slide.

### CROSS-REFERENCE TO RELATED APPLICATION(S)

This application is a continuation-in-part of PCT/US2024/034356, filed Jun. 17, 2024; which claim priority to U.S. Provisional Application Nos. 63/521,866, filed Jun. 19, 2023; and 63/562,960, filed Mar. 8, 2024. This application also claim priority to U.S. Provisional Application Nos. 63/729,909, filed Dec. 9, 2024, and 63/738,401, filed Dec. 23, 2024. The above identified applications are incorporated herein by reference in their entireties.

### FIELD OF THE INVENTION

The present invention relates to reagent-coated cover slips and methods of using them to carry out a reaction. The present invention provides a method using reagent-coated cover slips to carry out a test on a carrier slide. In particular, the present invention provides a method for detecting sperm DNA fragmentation (SDF) in a semen sample.

### BACKGROUND

Sperm DNA integrity is crucial for embryo quality, embryo implantation, and embryo development. Sperm DNA fragmentation (SDF) can be caused by extrinsic factors, such as radiation, environmental pollutants, and chemotherapeutics, as well as intrinsic factors, such as defective spermatogenesis, sperm apoptosis, and oxidative stress. SDF may cause male infertility, failed in vitro fertilization, and miscarriage. Therefore, the detection of SDF is important for fertility testing and assisted reproductive techniques.

Conventional methods for detecting SDF include sperm chromatin structure assay (SCSA), terminal deoxynucleotidyl transferase mediated dUTP nick end labeling (TUNEL) assay, DNA breakage detection-fluorescence in situ hybridization (DBD-FISH) test, comet assay (CA), and sperm chromatin dispersion (SCD) test.

Comet assay (CA), also known as single cell gel electrophoresis (SCGE), is a sensitive technique for detecting SDF. The procedures of CA involve embedding sperm cells in an agarose gel on a microscope slide, and then immersing the microscope slide in a lysis solution to break open the cell membrane and lyse the cellular proteins (e.g., protamine). Thereafter, the agarose gel is exposed to an electric field to attract negatively charged fragments of DNA toward the anode and form a comet-like structure. In the comet-like structure, the undamaged DNA nucleoid part is referred to as “head,” and the trailing damaged DNA streak is referred to as “tail.” After DNA staining with a fluorescent dye, the comet-like structure is visualized using a fluorescence microscope. Analysis of the comet tail can be performed by hand or with software, the fluorescence intensity of the comet tail indicating the extent of DNA damage. However, the operation of CA is complicated and time-consuming because of electrophoresis and software analysis processes, and thus CA cannot meet the needs of the industry.

SCD test is a modified halo assay that utilizes chemical methods to detect SDF. The procedures of the SCD test involve embedding sperm cells in an agarose gel, followed by DNA denaturation and deproteinization. Particularly, the double-stranded (DS) DNA of each sperm cell is denatured into a single-stranded (SS) DNA during the DNA denaturation. The nuclear protein (including protamine) of each sperm cell is lysed during the deproteinization. Therefore, DNA loops would be dispersed from the nuclear protein to the periphery of each sperm cell. After DNA staining with 4′,6-diamidino-2-phenylindole (DAPI) or the Diff-Quik reagent, the dispersed DNA loops are monitored by fluorescence or brightfield microscopy. The DNA loops of the sperm cell with DNA fragmentation are smaller than that of the sperm cell without DNA fragmentation, and more difficult to be stained. More specifically, the head of the sperm cell without DNA fragmentation shows as a large halo (i.e., the halo width is at least one-third of the diameter of the core head of the sperm cell). In contrast, the head of the sperm cell with DNA fragmentation shows as a small halo or no halo (i.e., the halo width is smaller than one-third of the diameter of the core head of the sperm cell). However, determining the halo width is difficult.

Methods for rapid and accurate detection of SDF have been reported in U.S. Pat. No. 11,644,455 and US Publication Nos. 2022/0195492; which are incorporated herein in their entities.

The Gram stain, the most widely used staining procedure in bacteriology, is a complex and differential staining procedure. Through a series of staining and decolorization steps, organisms in the Domain Bacteria are differentiated according to cell wall composition. Gram-positive bacteria have cell walls that contain thick layers of peptidoglycan (90% of cell wall); they stain purple. Gram-negative bacteria have cell walls with thin layers of peptidoglycan (10% of cell wall), and high lipid content; they stain pink. The performance of the Gram Stain on any sample requires four basic steps that include applying a primary stain (crystal violet) to a heat-fixed smear, followed by the addition of a mordant (Gram's Iodine), rapid decolorization with alcohol, acetone, or a mixture of alcohol and acetone and lastly, counterstaining with safranin.

Hematoxylin and eosin (H&E) stains are used in many areas of the histology laboratory, including frozen sections, fine needle aspirates, and paraffin fixed embedded tissues. The staining procedure for H&E follows a basic protocol: dewaxing, dehydration, hematoxylin, differentiation, bluing, eosin, dehydration, clearing, and cover-slipping. Hematoxylin is used to illustrate nuclear detail in cells. Depth of coloration is not only related to the amount of DNA in the nuclei but also to the length of time the sample spends in hematoxylin. Eosin is used as a counterstain that distinguishes between the cytoplasm and nuclei of cells. It is typically pink, with different shades of pink for different types of connective tissue fibers.

### BRIEF DESCRIPTION OF THE DRAWINGS

FIGS. 1A-1F illustrate one embodiment of the present method for detecting sperm double-stranded DNA fragmentation (SDF) in a semen sample.

FIGS. 2A-2F illustrate one embodiment of the present method for detecting sperm single-stranded DNA fragmentation (SDF) in a semen sample.

### DETAILED DESCRIPTION OF THE INVENTION

### Definitions

A “carrier slide”, as used herein, refers to a solid base where a sample, reagent, solution, and optionally other materials (e.g., gel) are added onto it. A carrier slide provides a platform (a base) for biochemical materials to be added on top of it and reactions to occur on it. A carrier slide can be made of any solid material such as glass, plastic, metal, ceramic, or quartz. Glass and plastic are preferred materials.

A “cover slip”, as used herein refers to a piece of solid material often thinner than the carrier slide, and has the same size or is smaller than the carrier slide. A cover slip is placed on top of the carrier slide to cover the materials on the carrier slide. A cover slip can be made of any solid material such as glass, plastic, metal, ceramic, or quartz. Glass and plastic are preferred materials. In one embodiment, the cover slip is transparent.

The present invention provides methods where sample cells are embedded, solidified, hardened, and immobilized on a carrier slide and a test is carried on the carrier slide. The present methods carry out one or more chemical reactions in the embedded sample cells on the carrier slide to result in a reaction product on the carrier slide.

In one aspect, the method uses one or more cover slips wherein one side of each cover slip is coated with one or more reagents to carry out a chemical reaction with sample cells embedded on a carrier slide. The cover slip is placed on top of the carrier slide with its coated side facing the carrier slide, to cause a reaction with the embedded cells.

In another aspect, the method adds one or more reagents to the sample cells embedded on a carrier slide to carry out a chemical reaction with the sample cells embedded on a carrier slide to cause a reaction product. Then one or more cover slips wherein one side of each cover slip is coated with one or more reagents is contacted with the reaction product to carry out further one or mor chemical reactions with sample cells embedded on a carrier slide.

In the present methods, sample cells are embedded, solidified, hardened, and immobilized on a carrier slide

The present methods simplify the reaction procedures using a carrier slide with immobilized sample cells.

### Methods of Using Reagent Coated Cover Slips

The present disclosure provides a method for carrying out a reaction with sample cells immobilized on a carrier slide. The method comprising the steps in the order of: (a) immobilizing sample cells on a carrier slide; (b) contacting the immobilized cells on the carrier slide with a coated side of a cover slip for a period of time to carry out a chemical reaction in the immobilized cells to result in a reaction product on the carrier slide, wherein the cover slip is coated with a reagent to react with the immobilized cells; (c) removing the cover slip from the immobilized cells without damaging the immobilized cells; and (d) obtaining the reaction product from cells.

In one embodiment, the sample cells are embedded in a gel and immobilized on the slide. For example, the sample cells are immobilized by embedding in a gel comprises acrylamide, acrylic acid, methacrylic acid, N-isopropylacrylamide (NIPAM), agarose, alginate, polyethylene glycol (PEG), or vinyl chloride.

In one embodiment, the sample cells are sperm cells from a semen sample.

In one embodiment, the method is a cell chromatin dispersion test.

In one embodiment, in step (b), the cover slip is coated with a DNA denaturing reagent and the chemical reaction comprises treating the sperm cell-embedding gel with the DNA denaturing reagent to denature DNAs in the sperm cells.

In one embodiment, in step (b), the cover slip is coated with a lysis reagent and the chemical reaction in step (b) comprises treating the sperm cell-embedding gel with the lysis reagent to lyse the nuclear proteins of the sperm cells embedded in the gel. For example, the lysis solution comprises 0.5-4 M urea and 0.05-0.5% w/v SDS.

In one embodiment, in step (b), the cover slip is a staining cover slip coated with one or more staining agents selected from the group consisting of: Wright-Giemsa solution, Diff-Quik staining, propidium iodide (PI), SYBR Green, 4′,6-diamidino-2-phenylindole (DAPI) staining, and acridine orange.

In one embodiment, after the step (d), further comprising a step (e): repeating the steps (b)-(d), for 1-5 times, each with a different cover slip coated with a different reagent.

In one embodiment, a first cover slip is coated with a DNA denaturing reagent to denature DNAs in the sperm cells, and then steps (b)-(d) are repeated with a second cover slip coated with a lysis reagent to lyse the nuclear proteins of the sperm cells embedded in the gel.

In one embodiment, steps (b)-(d) are repeated and the last cover slip in the last repeat is a staining cover slip coated with one or more staining agents.

If the immobilizing cells are dry, then an additional step of wetting the immobilized cells before contacting them with a coated cover slip is needed. If the immobilizing cells are wet, then this wetting step is not necessary. This applies when steps (b)-(d) are carried out once, or repeated 1-5 times.

In some embodiment, the method optionally further comprises a step after step (c) and before step (d): rinsing the reaction product with a rinsing solution and then removing the excess rinsing agent.

### Staining Reaction Product of Embedded Sample Cells

The present invention provides a method where a test is carried on a carrier slide and the final reaction product is stained with a dye-coated cover slip. The method uses a cover slip wherein one side of the cover slip is coated with one or more dyes to stain the products of a chemical reaction occurred on a carrier slide. The cover slip is placed on top of the carrier slide with coated side facing the carrier slide, to stain the product after the reaction on a carrier slide is complete. The present invention simplifies and accelerates the staining procedures on a carrier slide after the reaction is completed on the carrier slide.

### First Aspect

In a first aspect, the method for staining a reaction product includes embedding and immobilizing sample cells on a carrier slide, carrying out chemical reactions on the carrier slide, staining the reaction products with a dye-coated cover slip, and then removing the cover slip.

The method comprises the steps in the order of: (a) embedding sample cells on a carrier slide, (b) carrying out one or more chemical reactions in the embedded sample cells on the carrier slide to result in a reaction product on the carrier slide, (c) contacting the reaction product with a coated side of a cover slip for a period of time, wherein the coated side of the cover slip is coated with one or more staining agents, to stain the reaction product with the staining agents, (d) removing the cover slip from the stained reaction product without damaging the reaction product, and (e) obtaining the stained reaction product on the carrier slide.

In one embodiment, the method further comprises a step (b1) after step (b) and before step (c): removing excess liquid from the carrier slide such that no liquid flows on the carrier slide, and then applying an aqueous solution to wet the reaction product.

In one embodiment, the method further comprises a step (d1) after step (d) and before step (e): rinsing the stained reaction product to remove unbound staining agents and then drying the stained product.

If the embedded cells are dry, then an additional step of wetting the immobilized cells before contacting them with a coated cover slip is needed. If the immobilizing cells are wet, then this wetting step is not necessary.

In one embodiment, the method further comprises a step (f) after step (e): placing the stained reaction product on the carrier slide under microscope to examine the stained reaction product.

In one embodiment, the sample cells are embedded by a gel comprises acrylamide, acrylic acid, methacrylic acid, N-isopropylacrylamide (NIPAM), agarose, alginate, polyethylene glycol (PEG), or vinyl chloride.

In one embodiment, the one or more staining agents are selected from the group consisting of: Wright-Giemsa solution, Diff-Quik staining, propidium iodide (PI), SYBR Green, 4′,6-diamidino-2-phenylindole (DAPI) staining, and acridine orange.

In one embodiment, the sample cells are sperm cells from a semen sample.

In one embodiment, the method is a cell chromatin dispersion test, which is used for detecting sperm DNA fragmentation (SDF) in a semen sample by using a cover slip wherein one side of the cover slip is coated with one or more dyes that stain DNA. The cover slip is placed on top the carrier slide to stain DNA after the chemical reaction on a carrier slide is complete.

For detecting SDF, the chemical reaction in step (b) may comprise treating the sperm cell-embedding gel with a DNA denaturation solution to denature DNAs in the sperm cells.

For detecting SDF, the chemical reaction in step (b) may comprise treating the sperm cells-embedding gel with a lysis solution to lyse the nuclear proteins of the sperm cells embedded in the gel. In one embodiment, the lysis solution comprises 0.5-4M urea and 0.05-0.5% w/v SDS.

### Second Aspect

In a second aspect, the method for staining a reaction product includes embedding and immobilizing sample cells on a carrier slide, carrying out one or more chemical reactions on the carrier slide with one or more reagent-coated cover slips, removing the cover slips from the immobilized cells without damaging the cells, staining the reaction products with a dye-coated cover slip, and then removing the dye-coated cover slip without damaging the cells.

The method comprises the steps in the order of: (a) immobilizing sample cells on a carrier slide; (b) contacting the immobilized cells on the carrier slide with a coated side of a reaction cover slip to carry out a chemical reaction in the immobilized cells to result in a reaction product on the carrier slide, wherein the reaction cover slip is coated with a reagent to react with the immobilized cells; (c) removing the reaction cover slip from the immobilized cells without damaging the immobilized cells; (d) contacting the reaction product with a coated side of a staining cover slip, wherein the coated side of the staining cover slip is coated with one or more staining agents, to stain the reaction product with the staining agents, (e) removing the staining cover slip from the stained reaction product without damaging the reaction product, and (f) obtaining the stained reaction product on the carrier slide.

In the above method, the steps (b) and (c) may be repeated 1-5 times with different reaction cover slips coated with different reagents.

If the immobilized cells are dry, then an additional step of wetting the immobilized cells before contacting them with a cover slip coated with a reagent or a staining agent is needed. If the immobilizing cells are wet, then this wetting step is not necessary.

For detecting SDF, the reaction cover slip may be coated with a DNA denaturation solution to denature DNAs in the sperm cells.

For detecting SDF, the reaction cover slip may be coated with a lysis solution to lyse the nuclear proteins of the sperm cells embedded in the gel. In one embodiment, the lysis solution comprises 0.5-4M urea and 0.05-0.5% w/v SDS.

For detecting SDF, the steps (b) and (c) may be repeated one time with the first reaction cover slip coated with a DNA denaturation solution to denature DNAs in the sperm cells and with the second cover slip coated with a lysis solution to lyse the nuclear proteins of the sperm cells embedded in the gel.

### Sperm Dna Fragmentation (sdf) Detection Method

In one embodiment, the present method detects SDF in a semen sample. The method comprises the steps in the order of: (a) embedding and immobilizing the semen sample containing sperm cells in a gel on a carrier slide to obtain a sperm cell-embedding gel immobilized on a carrier slide; (b) treating the sperm cells-embedding gel with a lysis solution to lyse the nuclear proteins of the sperm cells embedded in the gel; (c) removing liquid from the gel; (d) obtaining a staining cover slip wherein one side of the cover slip is coated with one or more DNA staining agents, (e) applying an aqueous solution to the gel in step (c) with an aqueous solution (e.g., PBS or an ethanol aqueous solution) to wet the gel; (f) contacting the wet gel with the coated side of the cover slip to stain DNA with the staining agents; (g) removing the coverslip from the stained gel; (h) rinsing the stained gel to remove unbound staining agents; (i) drying the rinsed gel, and (j) examining the dried gel to observe the presence or the absence of a halo formation around a head of each sperm cell to determine SDF.

In one embodiment, the steps (b) and (c) of the method immediately above can be replaced by steps (b1) and (c1) as follows. (b1): contacting the immobilized cells on the carrier slide with a coated side of a reaction cover slip to carry out a chemical reaction in the immobilized cells to result in a reaction product on the carrier slide, wherein the reaction cover slip is coated with a lysis reagent to lyse the nuclear proteins of the sperm cells embedded in the gel; and (c1) removing the reaction cover slip from the immobilized cells without damaging the immobilized cells.

If the immobilizing cells are dry, then an additional step of wetting the immobilized cells before contacting them with a reaction cover slip is needed. If the immobilizing cells are wet, then this wetting step is not necessary. This applies when steps (b1)-(c1) are carried out only once, or repeated 1-5 times.

In one embodiment, the semen sample is a human semen sample.

In one embodiment, the semen sample is diluted with a diluent to have a sperm concentration ranging from 4×10⁶ cells/mL to 2.8×10⁷ cells/mL. Examples of diluents may include, but are not limited to, Earle's medium, human tubal fluid (HTF) medium, tris-buffered saline (TBS), phosphate-buffered saline (PBS), and saline.

DNA staining agents suitable for the present method include, but are not limited to Wright-Giemsa solution, Diff-Quik staining (0.4% Asure B plus 0.125% Eosin Y), propidium iodide (PI), SYBR Green, 4′,6-diamidino-2-phenylindole (DAPI) staining, and acridine orange.

### The First Sdf Embodiment

In a first embodiment, the present method detects sperm double-stranded DNA fragmentation (SDF) in a semen sample. After the reaction, the dried gel is examined to observe the presence or the absence of a halo formation around the heads of the sperm cells, wherein the presence of a halo formation is indicative of the presence of SDF.

In one embodiment, step (a) comprises embedding the semen sample containing semen cells in a gel comprising acrylamide, acrylic acid, methacrylic acid, N-isopropylacrylamide (NIPAM), alginate, or polyethylene glycol (PEG), so as to obtain a sperm cells-embedding gel. In a preferred embodiment, the gel is a polyacrylamide gel.

In one embodiment, step (b) comprises treating the sperm cells-embedded gel with a lysis solution comprising urea at a concentration ranging from 0.5 M to 4 M and sodium dodecyl sulfate (SDS) at a concentration ranging from 0.05% (w/v, g/mL) to 0.5% (w/v, g/mL), to lyse nuclear proteins of the sperm cells embedded in the gel. As an example, the lysis solution comprises 0.5-4M urea and 0.05-0.5% w/v SDS.

In another embodiment, steps (b) and (c) described above can be replaced by steps (b1) and (c1) as follows. (b1): contacting the immobilized sperm cells on the carrier slide with a coated side of a reaction cover slip to carry out a chemical reaction in the immobilized cells to result in a reaction product on the carrier slide, wherein the reaction cover slip is coated with a lysis solution comprising urea at a concentration ranging from 0.5 M to 4 M and sodium dodecyl sulfate (SDS) at a concentration ranging from 0.05% (w/v, g/mL) to 0.5% (w/v, g/mL), to lyse the nuclear proteins of the sperm cells embedded in the gel; and (c1) removing the reaction cover slip from the immobilized cells without damaging the immobilized cells. After (c1) or before (b1), if the immobilizing cells are dry, then an additional step of wetting the immobilized cells before contacting them with a reaction cover slip or a staining cover slip is needed. If the immobilizing cells are wet, then this wetting step is not necessary.

In one embodiment, in the lysis solution, sodium lauryl sulfate is used as an ionic surfactant, and urea is used as a protein denaturant. These two components can improve the lysis of protamine and thus the DNA loops can be easily released from the protamine to the periphery of the head of the sperm cell, and then be monitored as a halo via DNA staining, thereby reducing the time of lysis treatment (e.g., to less than 5 minutes). The lysis solution may further include an additional ionic or nonionic surfactant.

For example, the additional ionic surfactant may be selected from the group consisting of sodium deoxycholate, sodium cholate, sodium lauroyl sarcosinate, and any combination thereof.

In certain embodiments, the additional nonionic surfactant may be selected from the group consisting of Triton X-100, Nonoxynol-40 (NP-40), Pluronic F-127 (F-127), Tween-20,and any combination thereof. In an exemplary embodiment, the additional nonionic surfactant is Triton X-100.

In one embodiment, the lysis solution may further include an additional protein denaturant. Examples of the additional protein denaturant may include, but are not limited to, [(3-cholamidopropyl)dimethylammonio]-1-propanesulfonate hydrate, guanidinium chloride, and combinations thereof.

In one embodiment, the lysis solution may further include a reducing agent. Examples of the reducing agent may include, but are not limited to, dithiothreitol (DTT), β-mercaptoethanol, dithioerythritol (DTE), tributylphosphine (TBP), tris(2-carboxyethyl) phosphine (TCEP) hydrochloride, and combinations thereof. In an exemplary embodiment, the reducing agent is TCEP.

In one embodiment, the lysis solution may further include salts. Examples of the salts may include, but are not limited to, sodium chloride (NaCl), potassium chloride (KCl), and combinations thereof.

In one embodiment, the lysis solution may further include a titrant. Examples of the titrant may include, but are not limited to, sodium hydroxide (NaOH), hydrochloric acid (HCl), and a combination thereof.

In one embodiment, the lysis solution may further include 0.15 M to 3 M of NaCl, 0.05 M to 0.2 M of DTT, 0.1% (v/v) to 5% (v/v) of Triton X-100, and 0.01 M to 0.02 M of NaOH.

In an exemplary embodiment, the lysis solution includes 1 M urea, 0.05% (w/v) of SDS, 2.5 M NaCl, 0.1 M DTT, 1% (v/v) of Triton X-100, and 0.02 M NaOH. In another exemplary embodiment, the lysis solution includes 4 M urea, 0.05% (w/v) of SDS, 0.15 M NaCl, 0.2 M DTT, 0.5% (v/v) of Triton X-100, and 0.01 M NaOH. In yet another exemplary embodiment, the lysis solution includes 0.5 M urea, 0.5% (w/v) of SDS, 3 M NaCl, 0.05 M DTT, 5% (v/v) of Triton X-100, and 0.015 M NaOH.

The lysis solution may be adjusted to have a desired pH value. In certain embodiments, the lysis solution may have a pH value ranging from 7 to 9. In an exemplary embodiment, the lysis solution may have a pH value ranging from 7 to 8.2. In another exemplary embodiment, the lysis solution has a pH value of 7.5.

In one embodiment, step (a) of the method comprises embedding the semen sample containing semen cells in a polyacrylamide gel containing acrylamide at a concentration ranging from 3-22% (w/v), so as to obtain a sperm cells-embedded polyacrylamide gel, and step (b) of the method comprises the step of subjecting the sperm cells-embedded polyacrylamide gel to a lysis treatment with a lysis solution including urea at a concentration ranging from 0.5 M to 4 M and SDS at a concentration ranging from 0.05-0.5% (w/v).

In another embodiment, steps (b) and (c) described above can be replaced by steps (b1) and (c1) as follows. (b1): contacting the immobilized sperm cells on the carrier slide with a coated side of a reaction cover slip to carry out a chemical reaction in the immobilized cells to result in a reaction product on the carrier slide, wherein the reaction cover slip is coated with a lysis solution comprising urea at a concentration ranging from 0.5 M to 4 M and sodium dodecyl sulfate (SDS) at a concentration ranging from 0.05-0.5% (w/v), to lyse the nuclear proteins of the sperm cells embedded in the gel. (c1): removing the reaction cover slip from the immobilized cells without damaging the immobilized cells.

In certain embodiments, in step (a), the polyacrylamide gel contains acrylamide at a concentration ranging from 3-22% (w/v), 3-16% (w/v) or 4-16% (w/v). In one embodiment, the polyacrylamide gel is formed from acrylamide and bis-acrylamide in a ratio of acrylamide to bis-acrylamide ranging from 19:1 (w/w) to 37.5:1 (w/w).

In certain embodiments, in step (a), the polyacrylamide gel may have a pore size ranging from 3 nm to 10 nm or 3 nm to 9 nm.

After the gel is wetted by an aqueous solution, for example, phosphate-buffer saline PBS, in step (e), the wet gel is contacted with the coated side of the cover slip to stain DNA immediately when the gel is still wet. For example, the wet gel is contacted with the dye in less than 1 minute, less than 2 minutes, less than 5 minutes, less than 10 minutes, or less than 30 minutes.

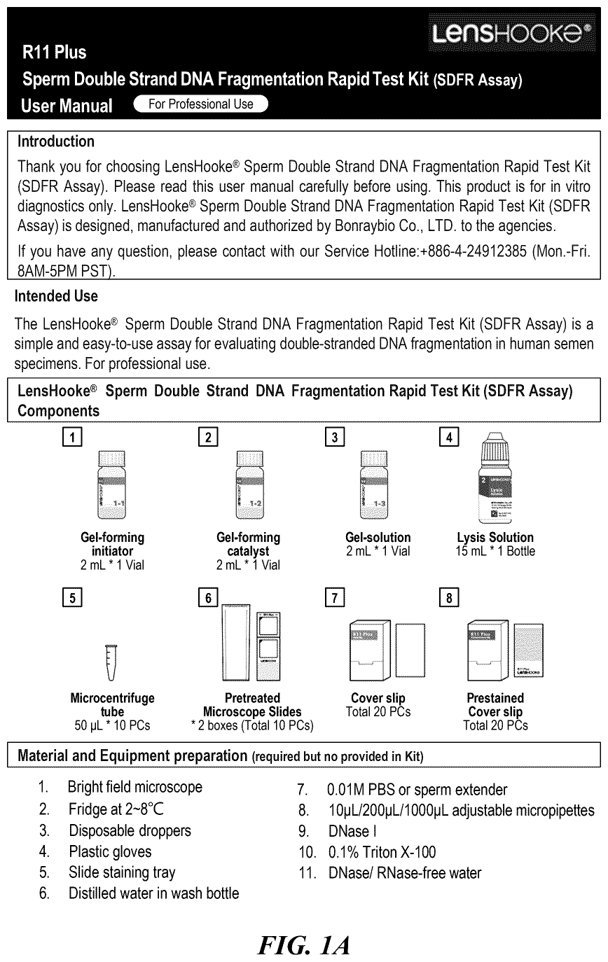

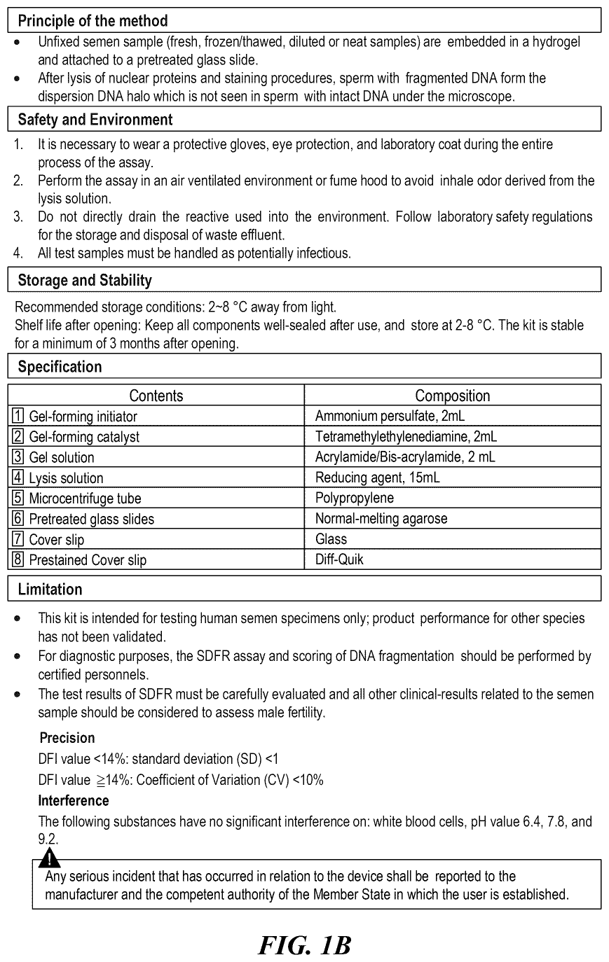

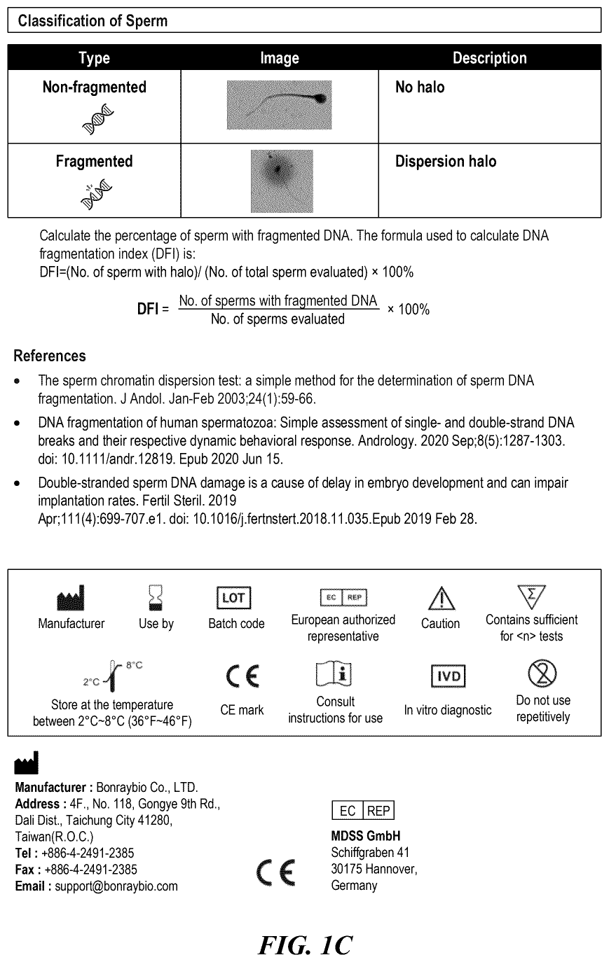

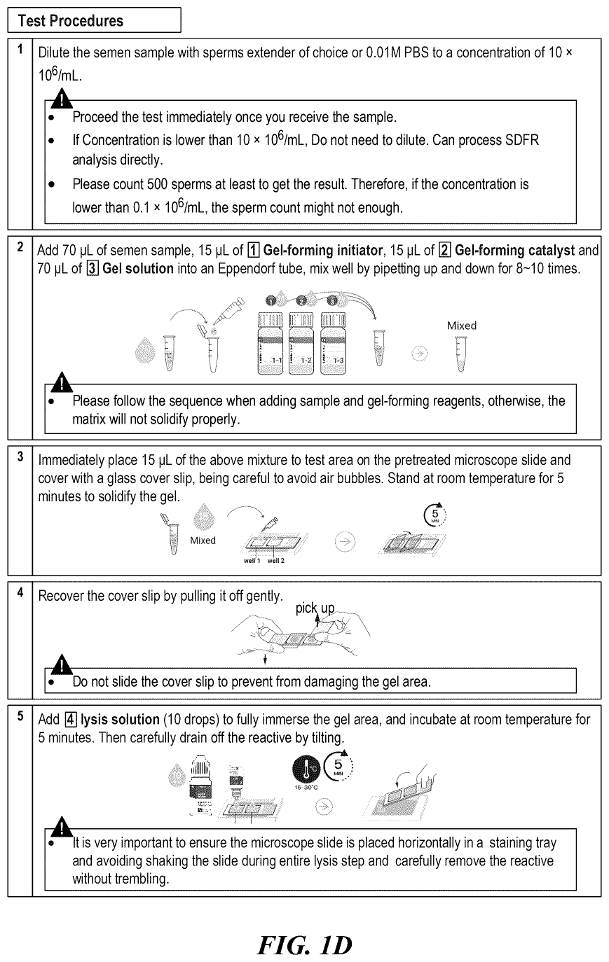

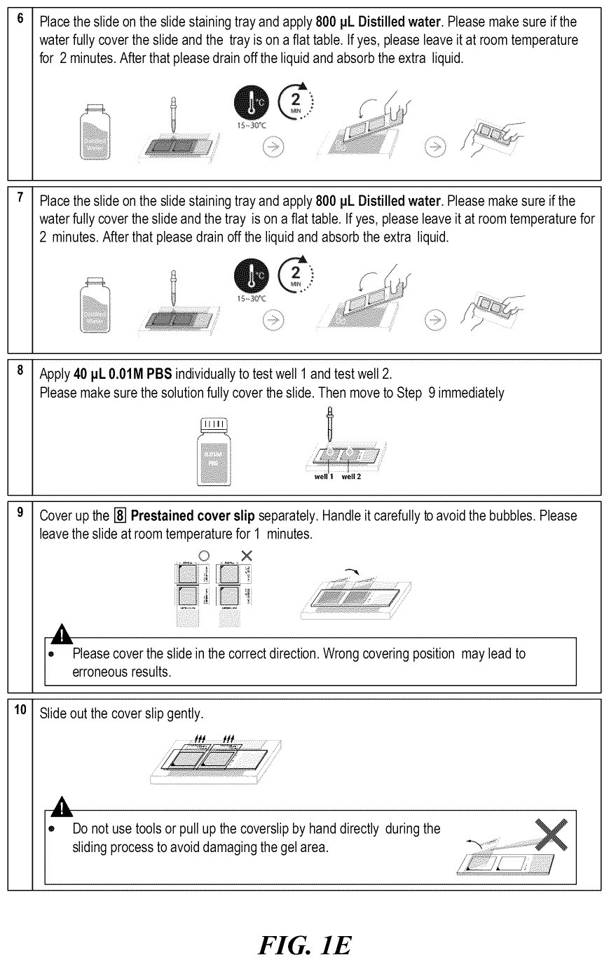

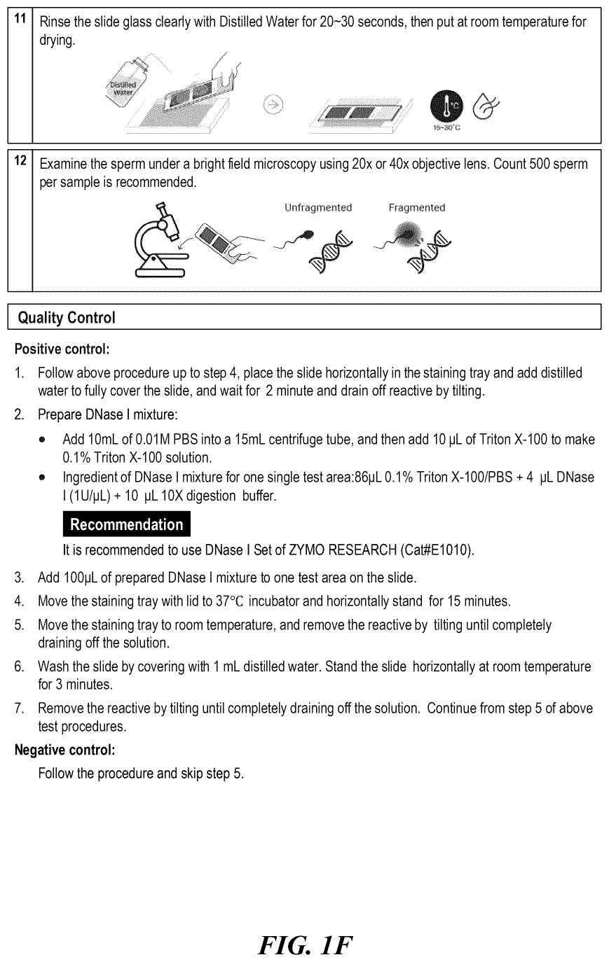

FIGS. 1A-1F illustrate one embodiment of the first aspect method for detecting sperm double-stranded DNA fragmentation (SDF) in a semen sample.

### The Second Sdf Embodiment

In a second embodiment, the present method detects sperm DNA fragmentation (SDF) in a semen sample comprising denaturing sperm DNAs to single-stranded DNAs.

The method comprises the steps in the order of: (a) embedding the semen sample containing sperm cells in a gel to obtain a sperm cell-embedding gel; (aa) treating the sperm cell-embedding gel with a DNA denaturing solution to denature DNAs in the sperm cells; (b) treating the sperm cells-embedding gel with a lysis solution to lyse the nuclear proteins of the sperm cells embedded in the gel; (c) removing liquid from the gel; (d) obtaining a cover slip wherein one side of the cover is coated with one or more DNA staining agents, (e) applying an aqueous solution to the gel in step (c) with an aqueous solution to wet the gel; (f) contacting the wet gel with the coated side of the cover slip to stain DNA with the staining agents; (g) removing the cover slip from the stained gel; (h) rinsing the stained gel to remove unbound staining agents; (i) drying the rinsed gel, and (j) examining the dried gel to observe the presence or the absence of a halo formation around a head of each sperm cell to determine SDF.

In one embodiment, step (aa) described immediately above can be replaced by steps (aa1) (aa2), (aa3 and (aa4)) as follows. (aa1): optionally wetting the gel. (aa2); contacting the immobilized and embedded sperm cells on the carrier slide with a coated side of a first reaction cover slip to denature DNA in the sperm cells on the carrier slide, wherein the first reaction cover slip is coated with a DNA denaturing agent. (aa3): removing the first reaction cover slip from the immobilized cells without damaging the immobilized cells. (aa4): optionally wetting the gel with a first aqueous solution.

In one embodiment, steps (b) and (c) described above can be replaced by steps (b1) as follows. (b1): contacting the immobilized and embedded sperm cells on the carrier slide with a coated side of a second reaction cover slip to lyse DNA in the sperm cells on the carrier slide, wherein the second reaction cover slip is coated with a lysis agent. (b2) removing the second reaction cover slip from the immobilized cells without damaging the immobilized cells. s

In one embodiment, the gel comprises agarose, acrylamide, alginate, or vinyl chloride.

In one embodiment, the gel may be an agarose gel. The agarose gel may have an agarose concentration ranging from 1-3% (w/v). In an exemplary embodiment, the agarose gel has an agarose concentration of 1.25% (w/v).

In one embodiment, the gel lysis solution including urea at a concentration ranging from 0.5 M to 4 M and sodium dodecyl sulfate at a concentration ranging from 0.05% (w/v) to 0.5% (w/v) to lyse the nuclear proteins of the sperm cells.

In one embodiment, prior to use for embedding the semen sample, the agarose gel may be melted at a temperature ranging from 95° C. to 100° C. using a microwave oven or a constant temperature water bath. In an exemplary embodiment, the agarose gel is melted at a temperature of 95° C.

In one embodiment, the DNA denaturing solution may be an acidic aqueous solution or an alkaline aqueous solution, and may have an equivalent concentration ranging from 0.05 N to 0.08 N. In certain embodiments, the DNA denaturing solution may have an equivalent concentration ranging from 0.06 N to 0.07 N.

In one embodiment, the DNA denaturing solution may be an acidic aqueous solution containing an acid selected from the group consisting of hydrochloric acid, acetic acid, nitric acid, sulfuric acid, and combinations thereof. In certain embodiments, the DNA denaturing solution is an acidic aqueous solution containing hydrochloric acid.

In one embodiment, the DNA denaturing solution may be an alkaline aqueous solution containing a base selected from the group consisting of sodium hydroxide, potassium hydroxide, calcium hydroxide, and combinations thereof. In certain embodiments, the DNA denaturing solution is an alkaline aqueous solution containing sodium hydroxide.

In the lysis solution, sodium lauryl sulfate, also referred to as sodium dodecyl sulfate (SDS) is used as an ionic surfactant, and urea is used as a protein denaturant. These two components improve the lysis of protamine and thus the DNA loops can be easily released from the protamine to the periphery of the head of the sperm cell, and then be monitored as a halo via DNA staining. Using SDS and urea in the lysis solution effectively reduces the time of lysis treatment (for example, to less than 5 minutes) and thus reducing the specimen uncertainty.

According to the present disclosure, the lysis solution may further include an additional ionic or nonionic surfactant.

In certain embodiments, the lysis solution may contain an ionic surfactant selected from the group consisting of sodium deoxycholate, sodium cholate, sodium lauroyl sarcosinate, and combinations thereof.

In certain embodiments, the lysis solution may contain nonionic surfactant selected from the group consisting of Triton X-100, Nonoxynol-40 (NP-40), Pluronic F-127 (F-127), Tween-20, and combinations thereof. In an exemplary embodiment, the nonionic surfactant is Triton X-100.

In certain embodiments, the lysis solution may further include an additional protein denaturant. Examples of the additional protein denaturant may include, but are not limited to, [(3-cholamidopropyl)dimethylammonio]-1-propanesulfonate hydrate, guanidinium chloride, and a combination thereof.

In certain embodiments, the lysis solution may further include a reducing agent. Examples of the reducing agent may include, but are not limited to, dithiothreitol (DTT), tris(2-carboxyethyl)phosphine (TCEP) hydrochloride, dithioerythritol (DTE), β-mercaptoethanol (β-ME), glutathione (GSH), dimercaprol, heparin, and combinations thereof. In an exemplary embodiment, the reducing agent is DTT or TCEP.

In certain embodiments, the lysis solution may further include salts. Examples of the salts may include, but are not limited to, sodium chloride (NaCl), potassium chloride (KCl), and a combination thereof.

In certain embodiments, the lysis solution may further include a pH titrant. Examples of the titrant may include, but are not limited to, sodium hydroxide (NaOH), hydrochloric acid (HCl), and a combination thereof.

In certain embodiments, the lysis solution may further include 0.15 M to 3 M of NaCl, 0.05 M to 0.2 M of DTT or TCEP, 0.1% (v/v) to 5% (v/v) of Triton X-100, and 0.01 M to 0.02 M of NaOH.

In an exemplary embodiment, the lysis solution includes 1 M urea, 0.05% (w/v, g/mL) of SDS, 2.5 M NaCl, 0.1 M DTT or 0.05 M TCEP, 1% (v/v) of Triton X-100, and 0.02 M NaOH. In another exemplary embodiment, the lysis solution includes 4 M urea, 0.05% (w/v, g/mL) of SDS, 0.15 M NaCl, 0.2 M DTT or 0.05 M TCEP, 0.5% (v/v) of Triton X-100, and 0.01 M NaOH. In yet another exemplary embodiment, the lysis solution includes 0.5 M urea, 0.5% (w/v, g/mL) of SDS, 3 M NaCl, 0.05 M DTT or TCEP, 5% (v/v) of Triton X-100, and 0.015 M NaOH.

According to the present disclosure, the lysis solution may be adjusted to have a desired pH value using the titrant. In certain embodiments, when the DNA denaturing solution is an acidic aqueous solution, the lysis solution may have a pH value ranging from 7.5 to 9.0. In certain embodiments, when the DNA denaturing solution is an alkaline aqueous solution, the lysis solution may have a pH value ranging from 5.5 to 7.0. In an exemplary embodiment, the lysis solution has a pH value ranging from 8.2 to 8.5.

### The Third Sdf Embodiment

In a third aspect, the present method provides another method comprising a step of denaturing sperm DNAs to single-stranded DNAs.

The method comprises: (a) subjecting an agarose solution to heat, followed by addition of a DNA denaturing solution and the semen sample containing semen cells, so as to form a mixture; (b) subjecting the mixture to a gel polymerization reaction, so as to obtain an agarose gel with the sperm cells containing denatured DNA embedded within; (c) subjecting the agarose gel to lysis with a lysis solution including urea at a concentration ranging from 0.5 M to 4 M and sodium dodecyl sulfate at a concentration ranging from 0.05-0.5% (w/v), so that nuclear proteins of the sperm cells embedded in the agarose gel are lysed; (d) removing liquid from the gel; (e) obtaining a cover slip wherein one side of the cover slip is coated with one or more DNA staining agents, (f) applying an aqueous solution to the gel in step (d) with an aqueous solution to wet the gel; (g) contacting the wet gel with the coated side of the cover slip to stain DNA with the staining agents; (h) removing the cover slip from the stained gel; (i) rinsing the stained gel to remove unbound staining agents; (j) drying the rinsed gel, and (k) examining the dried gel to observe the presence or the absence of a halo formation around a head of each sperm cell to determine SDF.

In step (a), the agarose solution may be further admixed with an acid-base indicator as described above before the heat treatment.

In step (a), the heat treatment may be conducted at a temperature ranging from 95° C. to 100° C. In an exemplary embodiment, the heating treatment is conducted at a temperature of 95° C.

In one embodiment, the method comprise the steps in the order of: (a) heating an agarose solution, followed by adding a DNA denaturing solution and a semen sample containing semen cells, to form a gel mixture; (b) polymerizing the gel mixture on a carrier slide, to obtain an agarose gel with the sperm cells containing denatured DNA embedded within, and optionally applying a first aqueous solution to wet the gel; (c) contacting the immobilized and embedded sperm cells on the carrier slide with a coated side of a reaction cover slip to lyse DNA in the sperm cells on the carrier slide, wherein one side of the reaction cover slip is coated with a lysis agent; (d) removing the reaction cover slip from the immobilized cells without damaging the immobilized cells; (e) optionally applying a second aqueous solution to the gel in step (d) with an aqueous solution to wet the gel; (f) contacting the wet gel with the coated side of a staining cover slip to stain DNA, wherein one side of the staining cover slip is coated with the staining agents; (g) removing the staining cover slip from the stained gel without damaging the cells; (h) rinsing and drying the gel, and (i) examining the dried gel to observe the presence or the absence of a halo formation around a head of each sperm cell to determine SDF.

### The Fourth Sdf Embodiment

In a fourth embodiment, the present method provides another method comprising denaturing sperm DNAs to single-stranded DNAs.

The method comprises: (a) admixing the semen sample containing sperm cells with a DNA denaturing solution and a gel-forming component, followed by subjecting a mixture thus obtained to a gel polymerization reaction, so as to obtain a gel with the sperm cells containing denatured DNA embedded within, the gel-forming component being selected from the group consisting of acrylamide, alginate, and vinyl chloride; (b) subjecting the gel to lysis with a lysis solution including urea at a concentration ranging from 0.5 M to 4 M and sodium dodecyl sulfate at a concentration ranging from 0.05-0.5% (w/v), so that nuclear proteins of the sperm cells embedded in the gel are lysed; (c) removing liquid from the gel; (d) obtaining a staining cover slip wherein one side of the cover slip is coated with one or more DNA staining agents, (e) applying an aqueous solution to the gel in step (c) to wet the gel; (f) contacting the wet gel with the coated side of the staining cover slip to stain DNA with the staining agents; (g) removing the staining cover slip from the stained gel; (h) rinsing the stained gel to remove unbound staining agents; (i) drying the rinsed gel, and (i) examining the dried gel to observe the presence or the absence of a halo formation around a head of each sperm cell to determine SDF.

In one embodiment, the method comprising the steps of: (a) admixing the semen sample containing sperm cells with a DNA denaturing solution and a gel-forming component selected from the group consisting of acrylamide, alginate, and vinyl chloride, followed by subjecting the mixture to a gel polymerization reaction on a carrier slide, to immobilize the gel on the carrier slide with the sperm cells containing denatured DNA embedded within, and optionally applying a first aqueous solution to wet the gel; (b) contacting the immobilized and embedded sperm cells on the carrier slide with a coated side of a reaction cover slip to lyse DNA in the sperm cells on the carrier slide, wherein the reaction cover slip is coated with a lysis agent; (c) removing the reaction cover slip from the immobilized cells without damaging the immobilized cells; (d) optionally applying a second aqueous solution to the gel to wet the gel; (e) contacting the wet gel with a coated side of the staining cover slip to stain DNA, wherein one side of the cover slip is coated with one or more DNA staining agents; (f) removing the staining cover slip from the stained gel; (g) rinsing and drying the stained gel; and (h) examining the dried gel to observe the presence or the absence of a halo formation around a head of each sperm cell to determine SDF.

In the methods of 2^{nd}, 3^{rd}, and the 4^{th} SDF embodiments, after the gel is wetted by an aqueous solution, for example, by an aqueous ethanol solution, the wet gel is contacted with the coated side of the staining cover slip to stain DNA immediately when the gel is still wet. For example, the wet gel is contacted with the dye in less than 1 minute, less than 2 minutes, less than 5 minutes, less than 8 minutes, or less than 10 minutes.

In the methods of 2^{nd}, 3^{rd}, and the 4^{th} SDF embodiment, the stained gel is examined to observe the presence or the absence of halo formation around heads of the sperm cells, wherein no halo formation or the presence of a halo having a halo width smaller than one third of a diameter of the corresponding sperm head is indicative of presence of sperm DNA fragmentation.

In the methods of 3^{rd}, and the 4^{th} aspects, common reagents are similar to those in the 2^{nd} aspect.

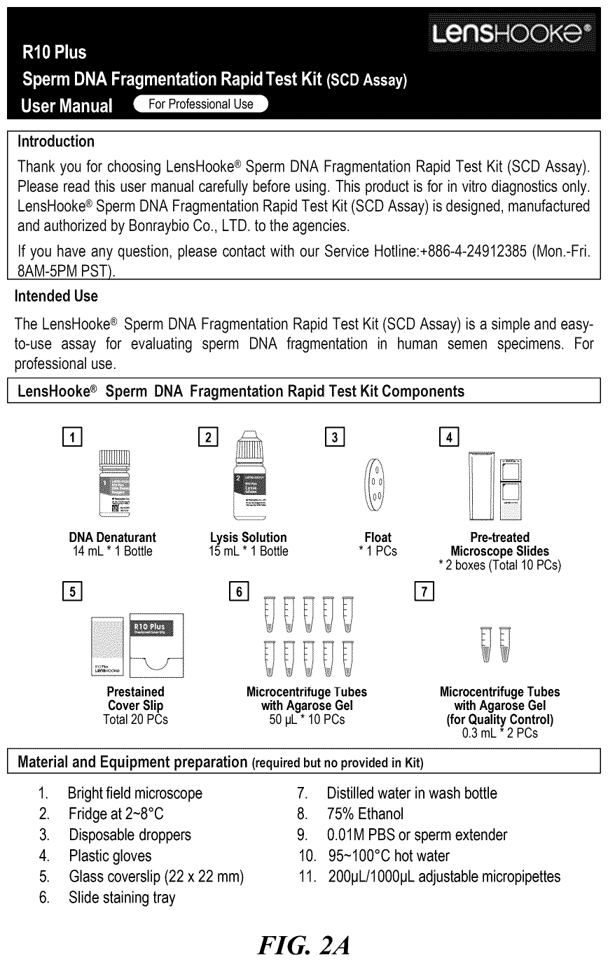

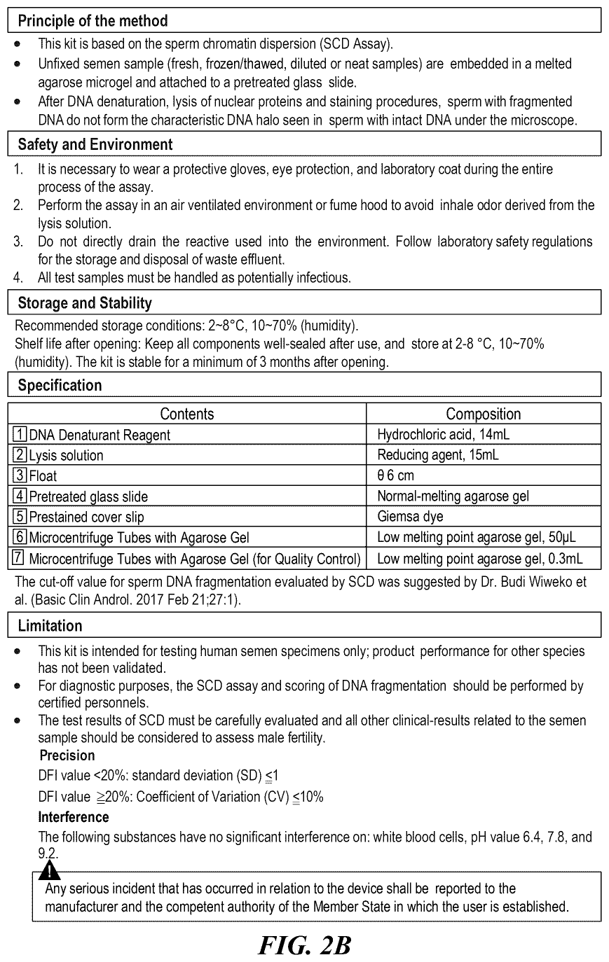

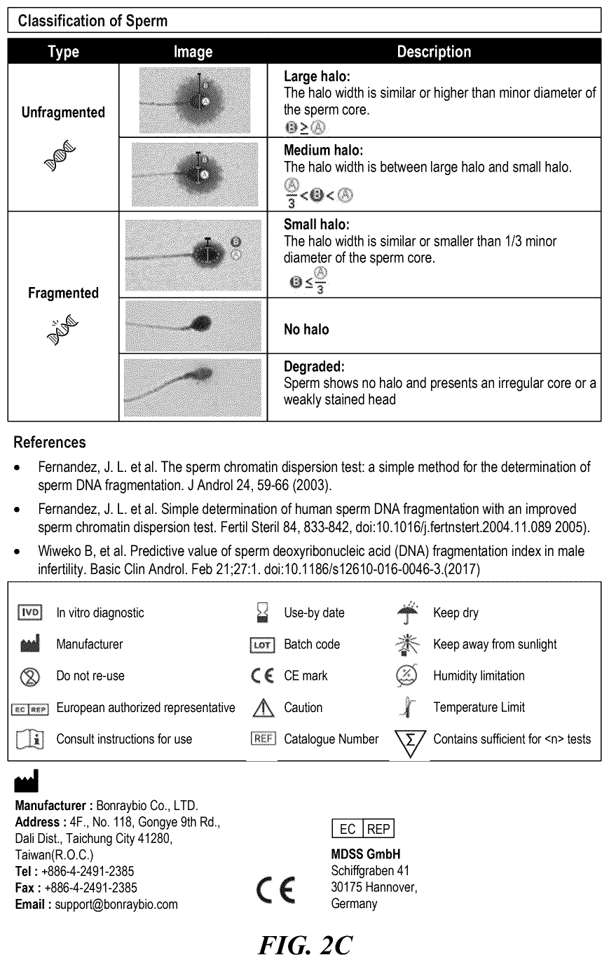

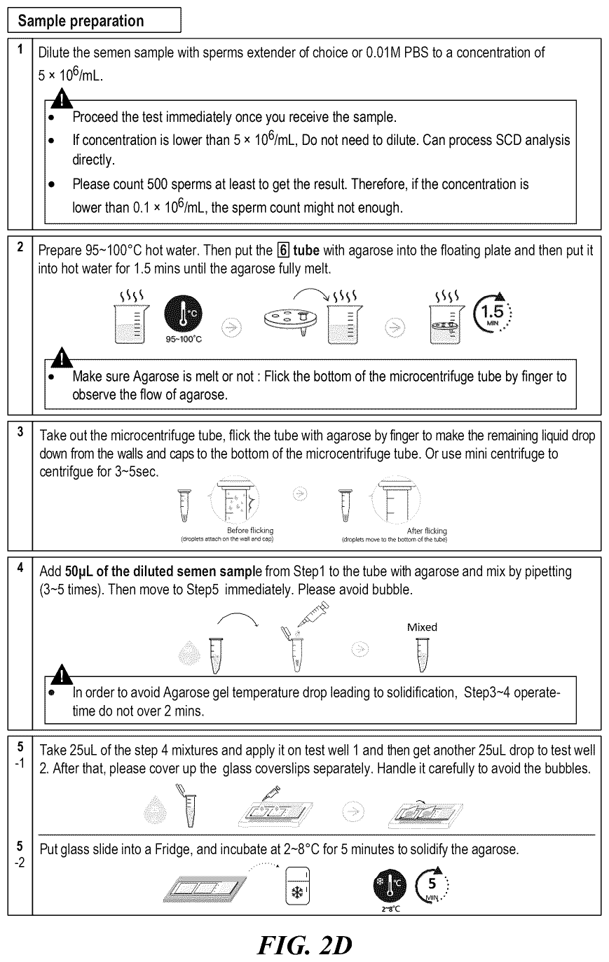

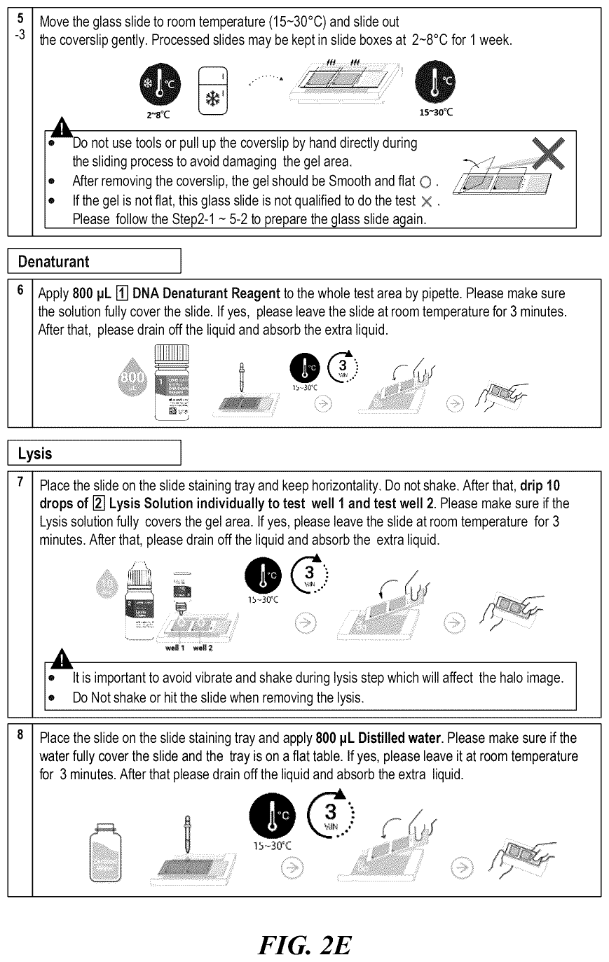

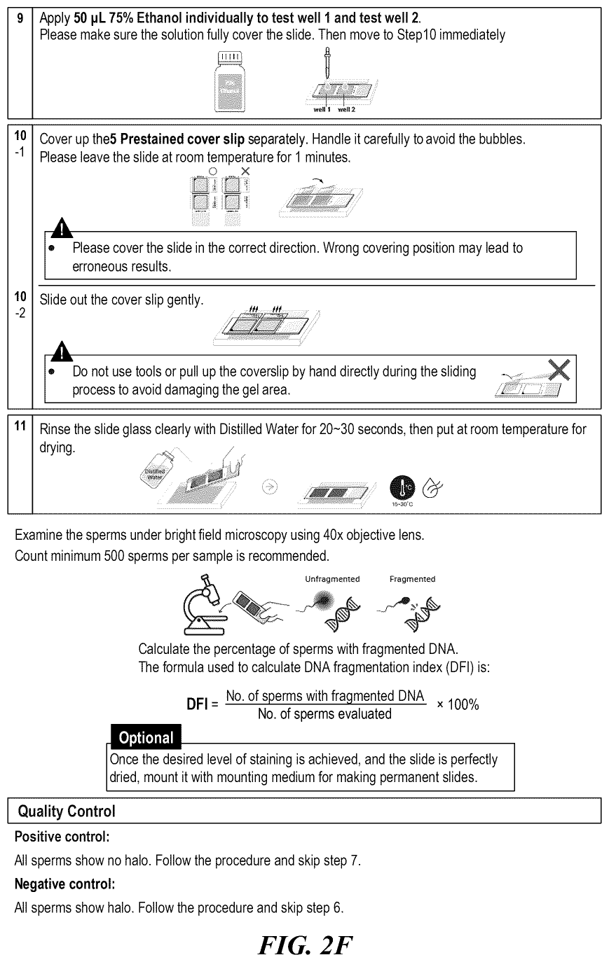

FIGS. 2A-2F illustrate the methods of second, third, and fourth embodiments for detecting sperm single-stranded DNA fragmentation (SDF) in a semen sample.

In all the present methods described above, after the step of observing the presence or the absence of halo formation around heads of the sperm cells, optionally the methods further comprise a step of calculating DNA fragmentation index (DFI) (%), which is the % of number of sperms with DNA fragmentation over the total number of sperms. In general, normal semen samples have DFI≤15%; abnormal semen samples have DFI≥30%. In between (15% [symbol] DFI [symbol] 30%) are considered borderline samples or threshold samples.

### Staining and Examining Morphology

The present invention provides a method for staining and examining the morphology of a reaction product. The method includes adhering a sample on a carrier slide, carrying out one or more reactions in the sample on the carrier slide, staining the reaction products with a dye-coated cover slip, and then examine the morphology of the sample without removing the cover slip. The method is useful for examining the morphology of bacteria, sperm cells and tissues.

In the method for staining and examining the morphology of a reaction product, the method comprises the steps in the order of: (a) adhering or immobilizing a sample on a carrier slide, wherein the sample is selected from the group consisting of tissues, body fluids, microorganisms, and cells; (b) carrying out one or more reactions in the sample on the carrier slide to result in a reaction product adhering to the carrier slide; (c) contacting the reaction product with a coated side of a cover slip for a period of time, wherein the coated side of the cover slip is coated with one or more staining agents, to stain the reaction product with the staining agents; and (d) placing the carrier slide, the stained reaction product, and the cover slip under microscope to examine the morphology of the stained reaction product.

In one embodiment, the step (a) adhering or immobilizing a sample on a carrier slide includes fixing a smear sample on a carrier slide.

In one embodiment, the step (a) adhering a sample on a carrier slide includes adhering a paraffin section of a tissue sample to the carrier slide via the cross-sectional surface interaction.

In one embodiment, the step (b) adhering a reaction product to the carrier slide includes dehydrating to immobilize the reaction product to the carrier slide.

In one embodiment, the present method is used for bacteria staining, for example, Gram stain. Gram stain is the most widely used staining procedure in bacteriology. For Gram stain, in step (a), the bacteria smear sample is applied onto a carrier slide, air-dried, and heat-fixed on the carrier slide. In step (b), the bacteria cells are reacted with crystal violet for staining, washed, stained with Gram's iodine, washed, reacted with an excess amount of a decolorizing agent (such as alcohol, acetone, or a mixture of alcohol and acetone) until the excess decolorizing agent becomes clear. In step (c), the reaction product of (b) is contacted with a coated side of a cover slip for a period of time (e.g., 30 seconds to 2 minutes), wherein the coated side of the cover slip is coated with a counterstain of safranin, to stain the reaction product with the staining agents. The stained reaction product together with the carrier slide and the cover slip are optionally washed and blot dried. In step (d), the carrier slide, the stained reaction product, and the cover slip are placed under microscope to examine the morphology of the stained reaction product. The gram-negative bacteria stain pink/red and gram-positive bacteria stain blue/purple.

In one embodiment, the present method is used for sperm morphology staining, for example, by Diff-quick staining, Papanicolaou staining, Shorr staining, HE staining, Wright staining, Wirght-Giemsa Staining, and then measurement of sperm head parameters and evaluation of sperm staining effects.

For example, in Diff-Quick staining, in step (a), the dried sperm smear of a semen sample is applied and fixed onto a carrier slide. In step (b), the smears are reacted with a solution containing methanol and eosin and washed. In step (c), the reaction product of (b) is contacted with a coated side of a cover slip for a period of time (e.g., 30 seconds to 2 minutes), wherein the coated side of the cover slip is coated with methylene blue, to stain the reaction product with the staining agents. The stained reaction product together with the carrier slide and the cover slip are washed and dried. In step (d), the carrier slide, the stained reaction product, and the cover slip are placed under microscope to examine the morphology (the highest sperm head length and width) of the stained reaction product.

For example, in Papanicolaou staining, in step (a), the sperm smear of a semen sample is applied onto a carrier slide, and fixed on the carrier slide. The smears were optionally rinsed with an aqueous ethanol solution and water. In step (b), the smears are reacted with hematoxylin staining solution (Harris) and washed. Then the smears are immersed in one or more aqueous ethanol solutions, stained with orange G staining solution, and immersed in an aqueous ethanol solution. In step (c), the reaction product of (b) is contacted with a coated side of a cover slip for a period of time (e.g., 30 seconds to 2 minutes), wherein the coated side of the cover slip is coated with EA36 staining solution (bright green and eosin), to stain the reaction product with the staining agents. The stained reaction product together with the carrier slide and the cover slip are optionally immersed in ethanol and blot dried. In step (d), the carrier slide, the stained reaction product, and the cover slip are placed under microscope to examine the morphology (the highest sperm head length and width) of the stained reaction product.

For example, in Shorr staining, in step (a), the dried sperm smear of a semen sample is applied onto a carrier slide, and fixed on the carrier slide in a solution containing methanol and triarylmethane and washed. In step (b), the smears are reacted with hematoxylin staining solution (Harris) and washed. Then the smears are immersed in an acidic ethanol solution, washed, and, and immersed in an aqueous ethanol solution. In step (c), the reaction product of (b) is contacted with a coated side of a cover slip for a period of time (e.g., 30 seconds to 2 minutes), wherein the coated side of the cover slip is coated with Shorr staining solution, to stain the reaction product with the staining agents. The stained reaction product together with the carrier slide and the cover slip are optionally immersed in ethanol and blot dried. In step (d), the carrier slide, the stained reaction product, and the cover slip are placed under microscope to examine the morphology (the highest sperm head length and width) of the stained reaction product.

In one embodiment, the present method is used for hematoxylin and eosin (H&E) staining of a tissue sample. H&E is the combination of two histological stains: hematoxylin and eosin. Hematoxylin stains cell nuclei a purplish blue, and eosin stains the extracellular matrix and cytoplasm pink, with other structures taking on different shades, hues, and combinations of these colors.

In the present H&E method, in step (a), a paraffin section of a piece of tissue is applied onto a carrier slide and adheres to the carrier slide. In step (b), the paraffin section is deparaffinized and rehydrated, e.g., with xylene, ethanol, and water; then reacted with hematoxylin stain, washed and blot dried. In step (c), the reaction product of (b) is contacted with a coated side of a cover slip for a period of time (e.g., 30 seconds to 2 minutes), wherein the coated side of the cover slip is coated with Eosin, to stain the reaction product with Eosin. The stained reaction product together with the carrier slide and the cover slip are washed and optionally dehydrated. In step (d), the carrier slide, the stained reaction product, and the cover slip are placed under microscope to examine the morphology of cell nuclei, cytoplasm, and the extracellular matrix of the stained reaction product.

It is to be understood that the foregoing describes preferred embodiments of the present invention and that modifications may be made therein without departing from the scope of the present invention as set forth in the claims.

What is claimed is:

1. A method for carrying out a reaction with sample cells, comprising the steps in the order of:
(a) immobilizing sample cells on a carrier slide;
(b) contacting the immobilized cells on the carrier slide with a coated side of a reaction cover slip to carry out a chemical reaction in the immobilized cells to result in a reaction product on the carrier slide, wherein the reaction cover slip is coated with a reagent to react with the immobilized cells;
(c) removing the cover slip from the immobilized cells without damaging the immobilized cells; and
(d) obtaining the reaction product from cells.

2. The method of claim 1, wherein the sample cells are embedded in a gel.

3. The method of claim 1, wherein the sample cells are solidified and hardened.

4. The method of claim 1, after the step (d), further comprising a step (e):
repeating the steps (b)-(d), for 1-5 times, each with a different cover slip with one side coated with a different reagent.

5. The method of claim 1, further comprising a step (e) after step (d):
(e) placing the reaction product on the carrier slide under microscope to examine the reaction product.

6. The method of claim 1, wherein the sample cells are immobilized by embedding in a gel comprises acrylamide, acrylic acid, methacrylic acid, N-isopropylacrylamide (NIPAM), agarose, alginate, polyethylene glycol (PEG), or vinyl chloride.

7. The method of claim 1, wherein the sample cells are sperm cells from a semen sample.

8. The method of claim 7, wherein the method is a cell chromatin dispersion test.

9. The method of claim 7, wherein the reaction cover slip is coated with a DNA denaturation solution to denature DNA in the immobilized sperm cells.

10. The method of claim 7, wherein the reaction cover slip is coated with a lysis solution to lyse the nuclear proteins of the cells embedded in the gel.

11. The method of claim 10, wherein the lysis solution comprises 0.5-4 M urea and 0.05-0.5% w/v SDS.

12. The method of claim 4, wherein the last cover slip is a staining cover slip coated with one or more staining agents selected from the group consisting of: Wright-Giemsa solution, Diff-Quik staining, propidium iodide (PI), SYBR Green, 4′,6-diamidino-2-phenylindole (DAPI) staining, and acridine orange.

13. A method for detecting sperm DNA fragmentation (SDF) in a semen sample, comprising the steps in the order of:
(a) embedding a semen sample containing sperm cells in a gel on a carrier slide to obtain a sperm cell-embedding gel immobilized on a carrier slide;
(b) optionally wetting the gel with a first aqueous solution;
(c) contacting the gel with a lysis cover slip coated with a lysis solution to lyse the nuclear proteins of the sperm cells embedded in the gel;
(d) removing the lysis cover ship from the gel without damaging the gel;
(e) optionally wetting the gel with a second aqueous solution;
(f) contacting the gel with a staining cover slip coated with one or more DNA staining agents to stain DNA;
(g) removing the staining coverslip from the stained gel without damaging the cells;
(h) rinsing the stained gel to remove unbound staining agents;
(i) drying the rinsed gel, and
(j) examining the dried gel to observe the presence or the absence of a halo formation around a head of each sperm cell to determine SDF.

14. The method of claim 13, wherein the sperm cells are solidified and hardened.

15. A method for detecting SDF in a semen sample, comprising the steps in the order of:
(a) embedding and immobilizing a semen sample containing sperm cells in a gel on a carrier slide to obtain a sperm cell-embedding gel immobilized on a carrier slide;
(b) contacting the immobilized sperm cells on the carrier slide with a coated side of a reaction cover slip to carry out a chemical reaction in the immobilized cells to result in a reaction product on the carrier slide, wherein the reaction cover slip is coated with a lysis solution comprising 0.5 M to 4 M of urea and 0.05-0.5% (w/v) sodium dodecyl sulfate (SDS), to lyse the nuclear proteins of the sperm cells embedded in the gel;
(c) removing the reaction cover slip from the immobilized cells without damaging the immobilized cells;
(d) obtaining a staining cover slip wherein one side of the cover slip is coated with one or more DNA staining agents;
(e) applying an aqueous solution to the gel in step (c) with an aqueous solution to wet the gel;
(f) contacting the wet gel with the coated side of the cover slip to stain DNA with the staining agents;
(g) removing the staining coverslip from the stained gel;
(h) rinsing the stained gel to remove unbound staining agents;
(i) drying the rinsed gel, and
(j) examining the dried gel to determine the presence or the absence of a halo formation around a head of each sperm cell to determine SDF.
